(19) United States
(12) Patent Application Publication
(10) Pub. No.: US 2024/0038369 A1
(43) Pub. Date: Feb. 1, 2024

(54) Systems and Methods for Transferring Multi-Frame Image Data

(71) Applicant: IDEXX Laboratories, Inc., Westbrook, ME (US)

(72) Inventors: Rahul Yeleti, Portland, ME (US); Katherine Healy, Portland, ME (US); Ben Schestopol, Scarborough, ME (US); Pavan Durgempudi, Portland, ME (US)

(21) Appl. No.: 18/359,056

(22) Filed: Jul. 26, 2023

Related U.S. Application Data
(60) Provisional application No. 63/392,853, filed on Jul. 27, 2022.

(51) Int. Cl. G16H 30/40; A61B 6/03; G06T 9/00; G16H 30/20

(52) U.S. Cl. CPC G16H 30/40; A61B 6/032; G06T 9/00; G16H 30/20

(57) ABSTRACT

Systems and methods for transferring multi-frame data to remote computing entities. Systems including dedicated communications for multi-frame data and other data, and data transfer methods. An example computer-implemented method includes receiving, at an agent module positioned at a primary location, multi-frame medical image data over an intranet data connection from an image acquisition device also positioned at the primary location, compressing at the agent module the multi-frame medical image data, sending the compressed data from the agent module to a server over a first data transmission via an Internet data connection, storing the compressed data at the server, sending the compressed data to a user terminal device, and receiving medical data at the server from one or more computing devices positioned at the primary location over a second data transmission via the Internet data connection.

### CROSS-REFERENCE TO RELATED APPLICATION

The present disclosure claims priority to U.S. application No. 63/392,853, filed on Jul. 27, 2022, the entire contents of which are herein incorporated by reference.

### TECHNICAL FIELD

The present disclosure relates to systems and methods for transferring multi-frame image data, such as computerized tomography (CT) data.

### BACKGROUND

In some applications, such as medical imaging, multiple images may be taken of a subject. As one example, CT machines combine X-ray and computer technology to produce multiple images of the inside of a subject, and can show detailed images of various body structures including bones, muscles, fat, organs, and blood vessels.

### SUMMARY

Many medical practices have limited computing resources to handle significant amounts of data, such as image data from multi-frame image devices/modalities. Accordingly, in many instances, medical practices utilize cloud computing, remote servers, and the like to process, store, and/or manage image data. However, the remote nature of the computing resources utilized can lead to long upload and download times, leading to increased diagnosis times. Accordingly, a need exists for improved systems and methods for transferring data.

In embodiments, systems of the present disclosure include at least two transmission connections between modalities and a remote computing entity. In some embodiments, one of the at least two transmission streams may be dedicated to transmitting multi-frame image data, while the other of the at least two transmission streams may be dedicated to transmitting data other than the multi-frame image data.

In some embodiments, systems of the present disclosure include agent modules in communication with a modality, such as a CT scanner. Multi-frame data is transferred in its entirety to the agent module, and then transferred in a compressed file to an image manager. The data in the compressed file can be transferred to a remote computing entity and accessed by a user at a customer terminal. By transferring the multi-frame image data to the agent module (in raw form) before transmission in compressed form to the image manager, overall transmission times can be reduced as compared to conventional configurations.

In some embodiments, image data is transmitted from a remote computing entity to a customer terminal in configurable packages that are less than the entirety of the multi-frame image data being sent. By transmitting data in configurable components less than the total size of the multi-frame image data being sent, users can begin viewing transmitted data before the entirety of the multi-frame image data is sent.

### BRIEF DESCRIPTION OF THE DRAWINGS

A detailed description of embodiments of the disclosure will be made with reference to the accompanying drawings, wherein like numerals designate corresponding parts in the figures:

FIG. 1 is a diagram of a computing system, according to one or more embodiments shown and described herein;

FIG. 2 is another diagram of the computing system of FIG. 1, according to one or more embodiments shown and described herein;

FIG. 3 shows a system for transmitting multi-frame image data, according to one or more embodiments shown and described herein;

FIG. 4 shows another system for transmitting multi-frame image data, according to one or more embodiments shown and described herein;

FIG. 5 shows a flowchart of an example of a computer-implemented method for transferring multi-frame medical image data to a server, according to one or more embodiments shown and described herein; and

FIG. 6 illustrates an example of a computing device, according to one or more embodiments shown and described herein.

### DETAILED DESCRIPTION

The present disclosure is generally directed to systems and methods for transmitting multi-frame image data. Medical practices, such as veterinary practices may take one or more images of a subject for analysis. As one example, CT scanners can be utilized to take multiple images of a subject for analysis. The multiple images from various devices, such as CT scanners, are referred to herein as multi-frame image data. The size of data generated by multi-frame image data devices/modalities, such as CT scanners, is significantly larger than the size of data generated by other diagnostic tools that generate single images or comparatively few images.

Embodiments of the present disclosure include computing systems including dedicated transmission pathways for multi-frame image modalities, separate from other transmission pathways. By providing dedicated transmission pathways, data can be transmitted more efficiently and quickly as compared to conventional computing systems. In some embodiments, computing systems according to the present disclosure include agent modules in communication with a multi-frame image modality. Multi-frame image data is transferred in its entirety to the agent module, and transferred in a compressed file to an image manager. The data in the compressed file can be transferred to a remote computing entity and accessed by a user at a customer terminal. By transferring the multi-frame image data to the agent module before transmission in compressed form to the image manager, transmission times can be reduced as compared to conventional configurations. In some embodiments image data is transmitted from a remote computing entity to a customer terminal in configurable packages that are less than the total size of the multi-frame image data being sent. By transmitting data in configurable packages less than the total size of the multi-frame image data being sent, users can begin viewing transmitted data before the entirety of the multi-frame data is sent. These and other embodiments will now be described with reference to the appended drawings.

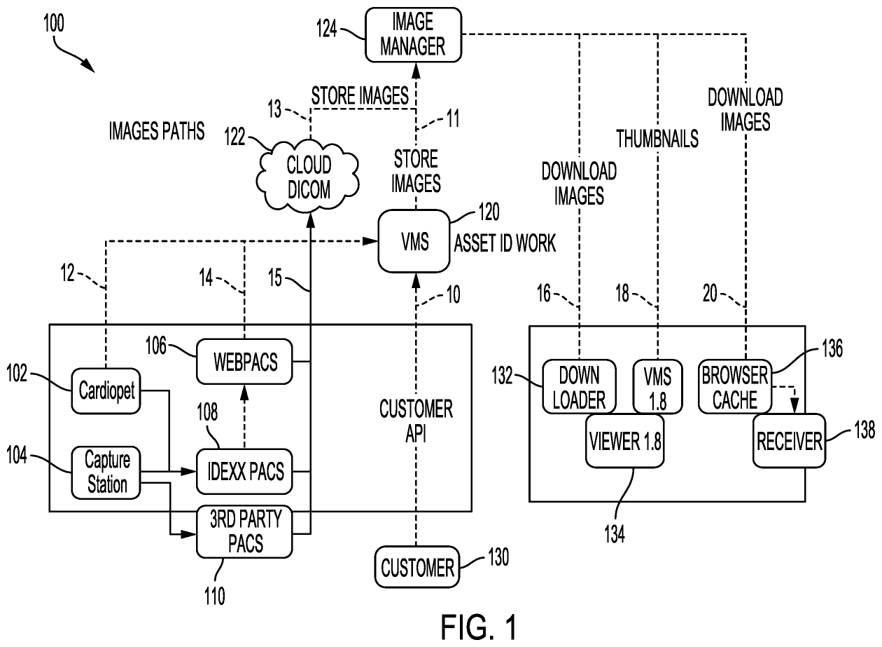

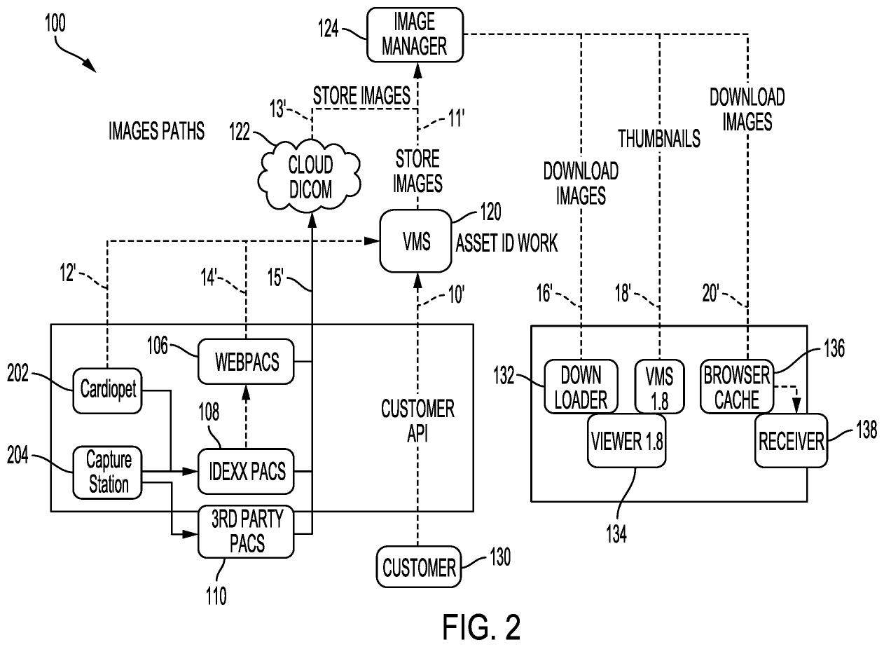

Referring to FIGS. 1 and 2, a computing system 100 is schematically depicted. In the depicted embodiment, the computing system 100 includes a customer terminal 130, a remote computing entity 120 (e.g., a VetMedStat (VMS) telemedicine provider), an image manager 124, multi-frame image modalities 102, 104, and other modalities 202, 204. The customer terminal 130 is communicatively coupled to the remote computing entity 120 via connections 10 (FIG. 1) and 10′ (FIG. 2). Multi-frame image modalities 102, 104 (FIG. 1) may include any suitable device for generating data comprising multiple images, for example and without limitation, CT scanners and the like. The multi-frame image modalities 102, 104 may be communicatively coupled to the remote computing entity 120 via one or more picture archiving and communication (PACS) modules 106, 108, 110, and connections 12, 14. In some embodiments, the multi-frame image modalities 102, 104 may be communicatively coupled to a cloud computing entity 122, such as via the one or more PACS modules 106, 108, 110 and connection 15.

Referring particularly to FIG. 1, in embodiments, the remote computing entity 120 and/or the cloud computing entity 122 may be communicatively coupled to the image manager 124, via connections 11 and 13, respectively. The image manager 124, in some embodiments, may be communicatively coupled to one or more viewers, browsers, and downloaders 132, 134, 136, 138 via connections 16, 18, and 20. While depicted as being separate from the customer terminal 130, it should be understood that the one or more viewers, browsers, downloaders 132, 134, 136, and 138 may be integrated within or communicatively coupled to the customer terminal 130.

By contrast and referring to FIG. 2, in some embodiments the other modalities 202 and 204 may be communicatively coupled to the remote computing entity 120 and the image manager 124 via connections 12′, 14′, which are separate from connections 12 (FIG. 1) and 14 (FIG. 1). As used herein the other modalities 202, 204 may include any devices other than the multi-frame modalities 102, 104, such as diagnostic analyzers, computing entities, and the like. Accordingly, data transferred to the remote computing entity 120 and/or the cloud computing entity 122 from the multi-frame image modalities 102, 104 passes along different connections as compared to the other modalities 202, 204. In this way, data traffic can be made more efficient as compared to conventional systems in which multi-frame image data and other data pass along the same connections. In particular, multi-frame image data may include multiple digital images, and may comprise a large size as compared to other data. Without being bound by theory, when large data sets (such as multi-frame image data) pass along the same connection as other data, delivery of the other data and the multi-frame image data may be delayed. For example, other data may be prevented from passing along the connection until all of the multi-frame image data has passed along the connection. By including dedicated connections for multi-frame image data and dedicated connections for other data between modalities and remote computing entities, both dedicated connections may operate in a manner as similar to a highway including a high-occupancy vehicle lane.

In some embodiments, the system 100 may include different connections 10 (FIG. 1) and 10′ (FIG. 2) between the customer terminal 130 and the remote computing entity 120 for multi-frame image data (connection 10) and other data (connection 10′). Similarly, in some embodiments, the system 100 may include different connections 16, 18, and 20 (FIG. 1) and 16′, 18′, and 20′ between the image manager 124 and the one or more viewers, browsers, downloaders 132, 134, 136, 138 for multi-frame image data (connections 16, 18, 20) and other data (connections 16′, 18′, and 20′).

In the system 100, any or all of the data connections include wired or wireless data communication (e.g., some components may be in wired Ethernet communication and others may use Wi-Fi or other wireless communication).

While the embodiment depicted in FIGS. 1 and 2 includes multi-frame image modalities 102, 104 and other modalities 202, 204, it should be understood that this is merely an example, and the system 100 may include any suitable number of multi-frame image modalities and other modalities.

In an example operation, the system 100 transfers multi-frame medical image data from the multi-frame image modalities 102, 104 to a server, which is the remote computing entity 120, the image manager 124, or a combination of the remote computing entity 120 and the image manager 124. The multi-frame image modalities 102, 104 and the PACS modules 106, 108, 110 are positioned at a primary location, and the PACS modules 106, 108, 110 receive multi-frame medical image data from the multi-frame image modalities 102, 104 over an intranet data connection. Following, the PACS modules 106, 108, 110 compress the multi-frame medical image data to form compressed data and send the compressed data to the server over a first data transmission (e.g., connections 12, 14) via an Internet data connection. In this example, the first data transmission is dedicated for carrying the compressed data. The other modalities 202, 204 (e.g., one or more computing devices positioned at the primary location) follow by sending medical data to the server over a second data transmission (e.g., connections 12′, 14′) via the Internet data connection. Thus, the system 100 includes different connections 10 (FIG. 1) and 10′ (FIG. 2) between the customer terminal 130 and the remote computing entity 120 for multi-frame image data (connection 10) and other data (connection 10′). Similarly, in some embodiments, the system 100 includes different connections 16, 18, and 20 (FIG. 1) and 16′, 18′, and 20′ between the image manager 124 and the one or more viewers, browsers, downloaders 132, 134, 136, 138 for multi-frame image data (connections 16, 18, 20) and other data (connections 16′, 18′, and 20′).

In embodiments, the primary location includes a veterinary clinic. As referred to herein, the term “veterinary clinics” includes any entity at which non-human animals receive medical care, and can include brick and mortar locations, mobile clinics, on-line virtual clinics, pop-up clinics, and the like.

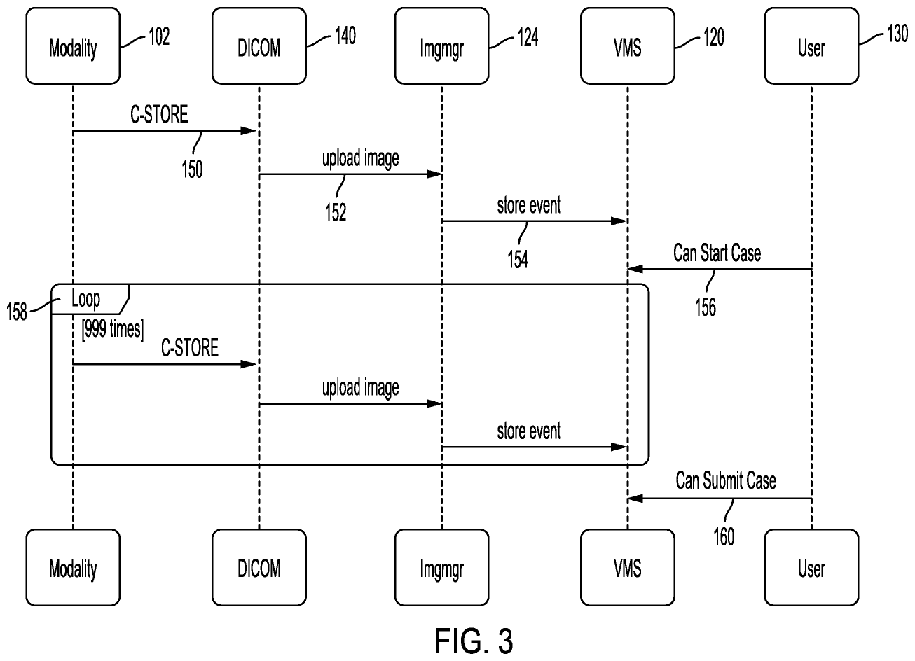

Referring to FIG. 3, one example of data transfer from the modality 102, such as a CT scanner or the like, to a user (e.g., a customer terminal 130 (FIGS. 1 and 2)) is schematically depicted. In embodiments, data representing one image is transferred to a module 140, such as a Digital Image and Communications in Medicine (DICOM) module, which is in communication with the modality 102. The data is transferred via an image data transmission 150. The modality 102 then uploads the image to the image manager (Imgmgr) 124 via an upload transmission 152. The image manager (Imgmgr) 124 then stores the image, shown by store event 154, in a remote computing entity (e.g., the VMS 120), and the user (e.g., via the customer terminal 130 (FIGS. 1 and 2)) is able to request access to the image to begin work on the image (e.g., view the image and/or start a case file for transmission to a remote entity shown by start case event 156). In this initial example in FIG. 3, a single image is uploaded for viewing.

In another scenario shown in FIG. 3, the multi-frame image data set includes 1000 total images, and each individual image follows the same workflow, i.e., transmission to the DICOM module, the Imgmgr, and to the remote computing entity (VMS). Thus, in FIG. 3, a loop 158 including the same workflow and data transmissions is performed 999 more times to complete the transfer of the 1000 total images after which the user may then access the case file (e.g., shown by submit case event 160). Thus, in examples in which the user wishes to create a case file for transmission to a remote entity, such as a telemedicine professional a specialist, or the like, all of the 1000 total images must be transmitted to the remote computing entity (VMS) before the case can be submitted to the telemedicine professional, specialist, or the like.

The example workflow in FIG. 3 can result in delays to accessing the case due to long upload times of images (i.e., a size of multi-frame images can be large) and the requirement to wait until all images are uploaded prior to being able to access the case. As a result, the example workflow in FIG. 3 can lead to increased diagnosis times.

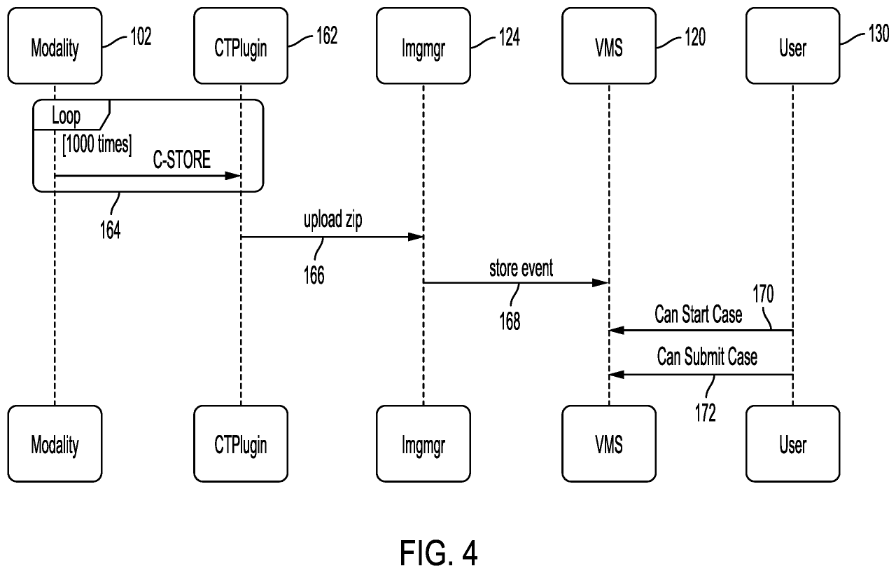

Referring to FIG. 4, another example of data transfer from the modality 102, such as a CT scanner or the like, to a user (e.g., the customer terminal 130 (FIGS. 1 and 2)) is schematically depicted. In the example depicted in FIG. 4, the system includes an agent module (e.g., CT Plugin 162) in communication with the modality 102. In embodiments, the agent module is positioned closer to the modality 102 as compared to the DICOM module (FIG. 3). In some embodiments, the agent module may be locally connected to and/or integrated with the modality 102. For instance, the agent module is a utility downloaded to the modality 102 for receiving and processing images.

In the example shown in FIG. 4, the entirety of the 1000 total images of the data set is transferred to the agent module and compressed (e.g., via zip operation or the like), as shown by loop transmission 164. Each image is sent sequentially one-by-one from the modality 102 to the CT Plugin 162. The compressed data including the entire 1000 total image data set may be uploaded to the image manager 124 as shown by upload zip message 166, and to the remote computing entity (e.g., VMS 120) as shown by store event 168. Because the agent module (CT Plugin 162) is positioned closer to the modality 102 as compared to the DICOM module (FIG. 3), the data set can be transferred to the agent module (CT Plugin 162) more quickly as compared to the DICOM module (FIG. 3), thereby reducing overall transfer time from the modality 102 to the remote computing entity (VMS 120). Following, the user via the customer terminal 130 can start a case or submit the case for viewing (as shown by events 170 and 172).

Thus, rather than sending raw image data to the image manager 124, as in the workflow shown in FIG. 3, data compression is performed at the modality 102 to generate a complete package to be sent.

In some embodiments, data is transferred to the customer terminal 130 (e.g., the one or more viewers, browsers, downloaders 132, 134, 136, 138) in configurable packages that are less than an entire data set, such that the user can begin viewing data before the entire data set is transferred to the customer terminal 130. For example, in some embodiments the configurable package may include 50 images, while the entire data set may include more than 50 images.

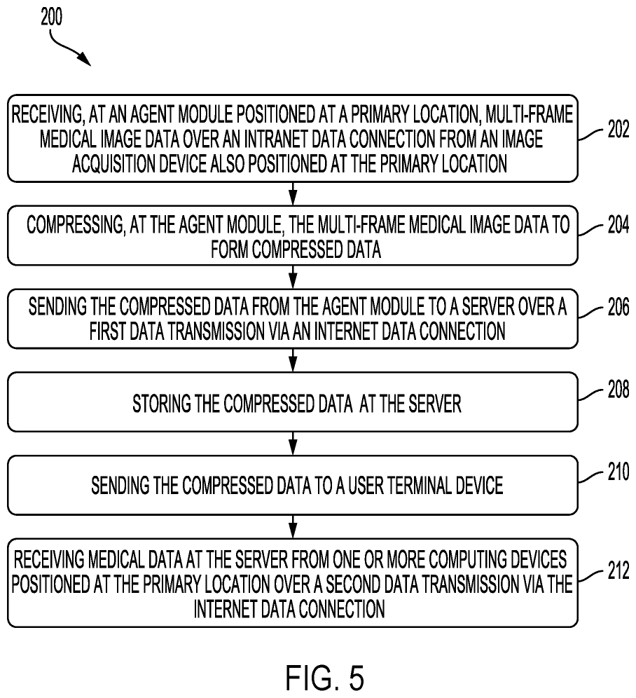

FIG. 5 shows a flowchart of an example of a computer-implemented method for transferring multi-frame medical image data to a server, according to an example implementation. Method 200 shown in FIG. 5 presents an example of a method that could be used with the system 100 shown in FIG. 1, or components of the system 100 shown in FIG. 1, for example. Further, devices or systems may be used or configured to perform logical functions presented in FIG. 5. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 5, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes receiving, at an agent module positioned at a primary location, multi-frame medical image data over an intranet data connection from an image acquisition device also positioned at the primary location. The multi-frame medical image data comprising a plurality of images of a subject. In one example, the image acquisition device is a computed tomography (CT) machine. In one example, functions of block 202 include receiving the multi-frame medical image data over a direct-wired connection between the agent module and the image acquisition device.

The agent module includes a software plugin installed on a computing device connected to the image acquisition device, for example, a controller of the image acquisition device. The agent module thus is located in close proximity to the image acquisition device enabling fast data transfer across the data connection.

In one example, the intranet connection takes the form of a local area network (LAN) connection.

At block 204, the method 200 includes compressing, at the agent module, the multi-frame medical image data to form compressed data. Compression is performed using loss-less data compression, for example.

The agent module assigns a file name to the compressed data. In one example, the file name is based on DICOM attributes of each of the images received. The file name optionally includes data appended to existing names of the images as well to—update to a user-defined naming e.

At block 206, the method 200 includes sending the compressed data from the agent module to a server over a first data transmission via an Internet data connection. The first data transmission is dedicated for carrying the compressed data. In one example, the multi-frame medical image data is received over an unencrypted data connection at the primary location, and functions of block 206 include sending the compressed data over an encrypted data connection.

In one example, the Internet data connection takes the form of a wide area network (WAN) connection.

At block 208, the method 200 includes storing the compressed data at the server.

At block 210, the method 200 includes sending the compressed data to a user terminal device. In one example, the user terminal device is positioned at the primary location.

The user terminal device requests a specific imaged procedure, and the server identifies and sends the corresponding compressed data file for the imaged procedure. In some examples, the user terminal device requests a specific image file from an imaged procedure using a subject identifier and image name. The server locates the corresponding compressed data file matching the subject identifier, decompresses (e.g., unzips) the compressed data file, and locates image data matching the image name to send to the user terminal device.

At block 212, the method 200 includes receiving medical data at the server from one or more computing devices positioned at the primary location over a second data transmission via the Internet data connection, and the first data transmission is separate from the second data transmission. In this example, the medical data does not include image data, and thus, the second data transmission is utilized for sending non-image data and the first data transmission is utilized for sending the image data. Separate data transmissions for image and non-image data enables the image data to be conveyed without delay, for example.

In one example, the method 200 also includes receiving, at the server, instructions to send one or more images of the compressed data to a third computing device.

In one example, the method 200 is executed after receiving a request through a radiology department's system according to a digital imaging and communications in medicine (DICOM) communications session. The DICOM standard specifies a set of information about patients, imaging equipment, procedures, and images. DICOM is hierarchically structured and has a Client-Server architecture. DICOM files contain both patient data and the image/pixel data. The patient data comes from the electronic medical record and image/pixel data is created by the radiology medical imaging devices.

In one example, the agent module receives frames of the multi-frame medical image data one by one in a sequential manner over a DICOM communications session with the image acquisition device. The plurality of images of the subject of the multi-frame medical image data comprise an imaged procedure. The agent module continues to receive from the image acquisition device the frames of the multi-frame medical image data one by one in the sequential manner until all the frames of the plurality of images have been received, and then the agent module compresses the multi-frame medical image data to form the compressed data by forming a single data package including all the plurality of images of the subject for the imaged procedure.

In other examples, the agent module compresses the multi-frame medical data to form the compressed data by forming a data package including less than all of the plurality of images of the subject for the imaged procedure. In this manner, portions of the imaged procedure are made available more quickly.

In operation, prior to the image acquisition device sending images to the agent module, a DICOM communication session is established. Once all images to be transferred are provided, then the DICOM communication session is closed by the image acquisition device following the DICOM protocol. The agent modules utilizes the DICOM communication session messaging to determine that the DICOM communication session is closed, at which point the agent module compresses all received images.

In some examples, the method 200 also includes caching the frames of the multi-frame medical image data at the agent module. Then after receiving a DICOM storage commitment request from the image acquisition device indicating that all of the plurality of images have been sent, functions include compressing, at the agent module, the multi-frame medical image data to form the compressed data, and sending a return confirmation message to the image acquisition device informing to delete local copies of the multi-frame medical image data.

In alternate examples, the method 200 includes caching the frames of the multi-frame medical image data at the agent module. Then based on the agent module experiencing a disconnection of the DICOM communications session with the image acquisition device prior to receiving a DICOM storage commitment request from the image acquisition device indicating that all of the plurality of images have been sent, functions include compressing, at the agent module, all the frames of the multi-frame medical image data that have been received to form the compressed data, and sending a return confirmation message to the image acquisition device informing to delete local copies of the multi-frame medical image data.

In further examples, the method 200 also includes receiving, at the agent module, a request for a modality worklist from the image acquisition device according to a DICOM protocol. The modality worklist includes an itemized list of imaging procedures scheduled to be performed by the image acquisition device. Following, functions include sending, by the agent module to the image acquisition device, the modality worklist over the intranet data connection. In this example, functions also can include determining an examination number for the imaged procedure from the itemized list of imaging procedures in the modality worklist, and associating the examination number with the multi-frame medical image data to link the plurality of images to the imaged procedure. An examination number is generated prior to imaging when an image order is created to synchronize image transfer using an accession number which links the radiology reports to a specific image study. The accession number is usually assigned by a hospital information system and can be repeating or unique depending on the system.

In yet one more example, the method 200 includes prior to receiving, at the agent module, the multi-frame medical image data from the image acquisition device, establishing a DICOM communications session between the agent module and the image acquisition device, and then subsequently receiving, at the agent module, frames of the multi-frame medical image data one by one in a sequential manner over the DICOM communications session with the image acquisition device until all the frames of the plurality of images have been received. The plurality of images of the subject comprise an imaged procedure, and a single data package is formed including all the plurality of images of the subject for the imaged procedure.

Throughout the disclosure, some components are described as “modules,” and such components include or take a form of a general purpose or special purpose hardware (e.g., general or special purpose processors), firmware, and/or software embodied in a non-transitory computer-readable (storage) medium for execution by one or more processors to perform described functionality.

Thus, the systems, devices, modules, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

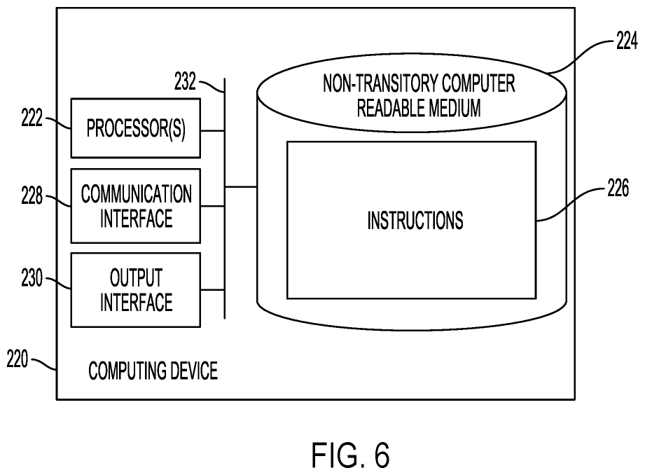

FIG. 6 illustrates an example of a computing device 220, according to an example implementation. The computing device 220 is representative of many components shown in FIGS. 1-2 including any of the customer terminal 130, the remote computing entity 120, the image manager 124, or the PACS modules 106, 108, 110, for example.

The computing device 220 includes one or more processor(s) 222, and non-transitory computer readable medium 224 having stored therein instructions 226 that when executed by the one or more processor(s) 222, causes the computing device 220 to perform functions for transferring multi-frame medical image data to a server, such as functions of the method 200 shown in FIG. 5, for example. To perform these functions, the computing device 220 also includes a communication interface 228, an output interface 230, and each component of the computing device 220 is connected to a communication bus 232. The computing device 220 may also include hardware to enable communication within the computing device 220 and between the computing device 220 and other devices (not shown). The hardware may include transmitters, receivers, and antennas, for example. The computing device 220 may further include a display.

In some embodiments, the communication interface 228 is a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 228 may be configured to receive input data from one or more devices, and may be configured to send output data to other devices.

The non-transitory computer readable medium 224 includes or takes the form of memory, such as one or more computer-readable storage media that can be read or accessed by the one or more processor(s) 222. The non-transitory computer readable medium 224 can include volatile and/or non-volatile storage components, such as optical, magnetic, organic or other memory or disc storage, which can be integrated in whole or in part with the one or more processor(s) 222. In some examples, the non-transitory computer readable medium 224 is implemented using a single physical device (e.g., one optical, magnetic, organic or other memory or disc storage unit), while in other examples, the non-transitory computer readable medium 224 is implemented using two or more physical devices. The non-transitory computer readable medium 224 thus is a computer readable storage, and the instructions 226 are stored thereon. The instructions 226 include computer executable code.

The one or more processor(s) 222 may be general-purpose processors or special purpose processors (e.g., digital signal processors, application specific integrated circuits, etc.). The one or more processor(s) 222 receive inputs from the communication interface 228 (e.g., x-ray images), and process the inputs to generate outputs that are stored in the non-transitory computer readable medium 224. The one or more processor(s) 222 are configured to execute the instructions 226 (e.g., computer-readable program instructions) that are stored in the non-transitory computer readable medium 224 and are executable to provide the functionality of the computing device 220 described herein.

The output interface 230 outputs information for transmission, reporting, or storage, and thus, the output interface 230 may be similar to the communication interface 228 and can be a wireless interface (e.g., transmitter) or a wired interface as well.

Implementations of this disclosure provide technological improvements that are particular to computer technology, for example, those concerning data transmission between computing devices. Computer-specific technological problems, such as timing of data transmission and dedicated transmission channels, can be wholly or partially solved by implementations of this disclosure. For example, implementation of this disclosure allows for the image modality to output images to a plugin, where the images are received over a short data transmission channel (e.g., intranet) and cached. Once all images of the procedure are received, the plugin compresses the images for a single data transmission over the Internet to a server. Any subsequent or additional medical data is also sent over the Internet, but using a separate data transmission.

As a result, different transmission channels are used for different types of data where image data and medical data are separately transmitted. A dedicated connection for multi-frame image data, which is compressed at the plugin before sending enables reduced data transmission times (as compared to other workflows that run a data transmission loop to complete transmission of all images).

As used herein, the term “exemplary” does not necessarily mean “preferred” and may simply refer to an example unless the context clearly indicates otherwise. Although the disclosure is not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like.

The embodiments disclosed herein are examples of the disclosure and may be embodied in various forms. For instance, although certain embodiments herein are described as separate embodiments, each of the embodiments herein may be combined with one or more of the other embodiments herein in any combination or any sub-combination. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Thus, examples of the present disclosure relate to enumerated clauses (ECs) listed below in any combination or any sub-combination.

EC 1 is a computer-implemented method for transferring multi-frame medical image data to a server, the method comprising receiving, at an agent module positioned at a primary location, multi-frame medical image data over an intranet data connection from an image acquisition device also positioned at the primary location, the multi-frame medical image data comprising a plurality of images of a subject, compressing, at the agent module, the multi-frame medical image data to form compressed data, sending the compressed data from the agent module to a server over a first data transmission via an Internet data connection, wherein the first data transmission is dedicated for carrying the compressed data, storing the compressed data at the server, sending the compressed data to a user terminal device, and receiving medical data at the server from one or more computing devices positioned at the primary location over a second data transmission via the Internet data connection, wherein the first data transmission is separate from the second data transmission.

EC 2 is the method of EC 1, further comprising receiving, at the server, instructions to send one or more images of the compressed data to a third computing device.

EC 3 is the method of any of ECs 1-2, wherein the image acquisition device is a computed tomography (CT) machine.

EC 4 is the method of any of ECs 1-3, wherein the user terminal device is positioned at the primary location.

EC 5 is the method of any of ECs 1-4, wherein receiving the multi-frame medical image data over the intranet data connection from the image acquisition device comprises receiving the multi-frame medical image data over a direct-wired connection between the agent module and the image acquisition device.

EC 6 is the method of any of ECs 1-5, wherein compressing the multi-frame medical data to form the compressed data comprises using loss-less data compression.

EC 7 is the method of any of ECs 1-6, wherein receiving, at the agent module, the multi-frame medical image data over the intranet data connection from the image acquisition device comprises receiving the multi-frame medical image data over an unencrypted data connection at the primary location, and sending the compressed data from the agent module to the server over the first data transmission via the Internet data connection comprises sending the compressed data over an encrypted data connection.

EC 8 is the method of any of ECs 1-7, wherein receiving the multi-frame medical image data comprises receiving, at the agent module, frames of the multi-frame medical image data one by one in a sequential manner over a digital imaging and communications in medicine (DICOM) communications session with the image acquisition device.

EC 9 is the method of any of ECs 1-8, wherein the plurality of images of the subject comprise an imaged procedure, the agent module continues to receive from the image acquisition device the frames of the multi-frame medical image data one by one in the sequential manner until all the frames of the plurality of images have been received, and compressing the multi-frame medical image data to form the compressed data comprises forming a single data package including all the plurality of images of the subject for the imaged procedure.

EC 10 is the method of any of ECs 1-9, wherein the plurality of images of the subject comprise an imaged procedure, and compressing the multi-frame medical data to form the compressed data comprises forming a data package including less than all of the plurality of images of the subject for the imaged procedure.

EC 11 is the method of any of ECs 1-10, further comprising caching the frames of the multi-frame medical image data at the agent module, and after receiving a DICOM storage commitment request from the image acquisition device indicating that all of the plurality of images have been sent: compressing, at the agent module, the multi-frame medical image data to form the compressed data, and sending a return confirmation message to the image acquisition device informing to delete local copies of the multi-frame medical image data.

EC 12 is the method of any of ECs 1-11, further comprising caching the frames of the multi-frame medical image data at the agent module, and based on the agent module experiencing a disconnection of the DICOM communications session with the image acquisition device prior to receiving a DICOM storage commitment request from the image acquisition device indicating that all of the plurality of images have been sent: compressing, at the agent module, all the frames of the multi-frame medical image data that have been received to form the compressed data, and sending a return confirmation message to the image acquisition device informing to delete local copies of the multi-frame medical image data.

EC 13 is the method of any of ECs 1-12, further comprising receiving, at the agent module, a request for a modality worklist from the image acquisition device according to a digital imaging and communications in medicine (DICOM) protocol, wherein the modality worklist includes an itemized list of imaging procedures scheduled to be performed by the image acquisition device, and sending, by the agent module to the image acquisition device, the modality worklist over the intranet data connection.

EC 14 is the method of any of ECs 1-13, wherein the plurality of images of the subject comprise an imaged procedure, and the method further comprises determining an examination number for the imaged procedure from the itemized list of imaging procedures in the modality worklist, and associating the examination number with the multi-frame medical image data to link the plurality of images to the imaged procedure.

EC 15 is the method of any of ECs 1-14, further comprising prior to receiving, at the agent module, the multi-frame medical image data from the image acquisition device, establishing a digital imaging and communications in medicine (DICOM) communications session between the agent module and the image acquisition device, subsequently receiving, at the agent module, frames of the multi-frame medical image data one by one in a sequential manner over the DICOM communications session with the image acquisition device until all the frames of the plurality of images have been received, and wherein the plurality of images of the subject comprise an imaged procedure, and compressing the multi-frame medical image data to form the compressed data comprises forming a single data package including all the plurality of images of the subject for the imaged procedure.

EC 16 is a system for transferring multi-frame medical image data to a server, the system comprising a server coupled to an Internet data connection, an agent module positioned at a primary location receiving multi-frame medical image data over an intranet data connection from an image acquisition device also positioned at the primary location, the multi-frame medical image data comprising a plurality of images of a subject, wherein the agent module compresses the multi-frame medical image data to form compressed data and sends the compressed data to the server over a first data transmission via the Internet data connection, wherein the first data transmission is dedicated for carrying the compressed data, and one or more computing devices positioned at the primary location sending medical data to the server over a second data transmission via the Internet data connection, wherein the first data transmission is separate from the second data transmission.

EC 17 is the system of EC 16, wherein the one or more computing devices comprise a picture archiving and communication system (PACS).

EC 18 is the system of any of ECs 16-17, wherein the agent module receives frames of the multi-frame medical image data one by one in a sequential manner over a digital imaging and communications in medicine (DICOM) communications session with the image acquisition device.

EC 19 is the system of any of ECs 16-18, wherein the plurality of images of the subject comprise an imaged procedure, the agent module continues to receive from the image acquisition device the frames of the multi-frame medical image data one by one in the sequential manner until all the frames of the plurality of images have been received, and the agent module compresses the multi-frame medical data to form the compressed data including a single data package with all the plurality of images of the subject for the imaged procedure.

EC 20 is the system of any of ECs 16-19, wherein the agent module caches the frames of the multi-frame medical image data at the agent module, and after receiving a DICOM storage commitment request from the image acquisition device indicating that all of the plurality of images have been sent: compresses, at the agent module, the multi-frame medical image data to form the compressed data, and sends a return confirmation message to the image acquisition device informing to delete local copies of the multi-frame medical image data.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A computer-implemented method for transferring multi-frame medical image data to a server, the method comprising:
receiving, at an agent module positioned at a primary location, multi-frame medical image data over an intranet data connection from an image acquisition device also positioned at the primary location, the multi-frame medical image data comprising a plurality of images of a subject;
compressing, at the agent module, the multi-frame medical image data to form compressed data;
sending the compressed data from the agent module to a server over a first data transmission via an Internet data connection, wherein the first data transmission is dedicated for carrying the compressed data;
storing the compressed data at the server;
sending the compressed data to a user terminal device; and
receiving medical data at the server from one or more computing devices positioned at the primary location over a second data transmission via the Internet data connection, wherein the first data transmission is separate from the second data transmission.

2. The computer-implemented method of claim 1, further comprising:
receiving, at the server, instructions to send one or more images of the compressed data to a third computing device.

3. The computer-implemented method of claim 1, wherein the image acquisition device is a computed tomography (CT) machine.

4. The computer-implemented method of claim 1, wherein the user terminal device is positioned at the primary location.

5. The computer-implemented method of claim 1, wherein receiving the multi-frame medical image data over the intranet data connection from the image acquisition device comprises receiving the multi-frame medical image data over a direct-wired connection between the agent module and the image acquisition device.

6. The computer-implemented method of claim 1, wherein compressing the multi-frame medical data to form the compressed data comprises using loss-less data compression.

7. The computer-implemented method of claim 1, wherein
receiving, at the agent module, the multi-frame medical image data over the intranet data connection from the image acquisition device comprises receiving the multi-frame medical image data over an unencrypted data connection at the primary location, and
sending the compressed data from the agent module to the server over the first data transmission via the Internet data connection comprises sending the compressed data over an encrypted data connection.

8. The computer-implemented method of claim 1, wherein receiving the multi-frame medical image data comprises receiving, at the agent module, frames of the multi-frame medical image data one by one in a sequential manner over a digital imaging and communications in medicine (DICOM) communications session with the image acquisition device.

9. The computer-implemented method of claim 8, wherein:
the plurality of images of the subject comprise an imaged procedure;
the agent module continues to receive from the image acquisition device the frames of the multi-frame medical image data one by one in the sequential manner until all the frames of the plurality of images have been received; and
compressing the multi-frame medical image data to form the compressed data comprises forming a single data package including all the plurality of images of the subject for the imaged procedure.

10. The computer-implemented method of claim 8, wherein:
the plurality of images of the subject comprise an imaged procedure; and
compressing the multi-frame medical data to form the compressed data comprises forming a data package including less than all of the plurality of images of the subject for the imaged procedure.

11. The computer-implemented method of claim 8, further comprising:
caching the frames of the multi-frame medical image data at the agent module; and
after receiving a DICOM storage commitment request from the image acquisition device indicating that all of the plurality of images have been sent:
compressing, at the agent module, the multi-frame medical image data to form the compressed data; and
sending a return confirmation message to the image acquisition device informing to delete local copies of the multi-frame medical image data.

12. The computer-implemented method of claim 11, further comprising:
caching the frames of the multi-frame medical image data at the agent module; and
based on the agent module experiencing a disconnection of the DICOM communications session with the image acquisition device prior to receiving a DICOM storage commitment request from the image acquisition device indicating that all of the plurality of images have been sent:
compressing, at the agent module, all the frames of the multi-frame medical image data that have been received to form the compressed data; and
sending a return confirmation message to the image acquisition device informing to delete local copies of the multi-frame medical image data.

13. The computer-implemented method of claim 1, further comprising:
receiving, at the agent module, a request for a modality worklist from the image acquisition device according to a digital imaging and communications in medicine (DICOM) protocol, wherein the modality worklist includes an itemized list of imaging procedures scheduled to be performed by the image acquisition device; and
sending, by the agent module to the image acquisition device, the modality worklist over the intranet data connection.

14. The computer-implemented method of claim 13, wherein the plurality of images of the subject comprise an imaged procedure, and the method further comprises:
determining an examination number for the imaged procedure from the itemized list of imaging procedures in the modality worklist; and
associating the examination number with the multi-frame medical image data to link the plurality of images to the imaged procedure.

15. The computer-implemented method of claim 1, further comprising:
prior to receiving, at the agent module, the multi-frame medical image data from the image acquisition device, establishing a digital imaging and communications in medicine (DICOM) communications session between the agent module and the image acquisition device;
subsequently receiving, at the agent module, frames of the multi-frame medical image data one by one in a sequential manner over the DICOM communications session with the image acquisition device until all the frames of the plurality of images have been received; and
wherein the plurality of images of the subject comprise an imaged procedure, and compressing the multi-frame medical image data to form the compressed data comprises forming a single data package including all the plurality of images of the subject for the imaged procedure.

16. A system for transferring multi-frame medical image data to a server, the system comprising:
a server coupled to an Internet data connection;
an agent module positioned at a primary location receiving multi-frame medical image data over an intranet data connection from an image acquisition device also positioned at the primary location, the multi-frame medical image data comprising a plurality of images of a subject, wherein the agent module compresses the multi-frame medical image data to form compressed data and sends the compressed data to the server over a first data transmission via the Internet data connection, wherein the first data transmission is dedicated for carrying the compressed data; and
one or more computing devices positioned at the primary location sending medical data to the server over a second data transmission via the Internet data connection, wherein the first data transmission is separate from the second data transmission.

17. The system of claim 16, wherein the one or more computing devices comprise a picture archiving and communication system (PACS).

18. The system of claim 16, wherein the agent module receives frames of the multi-frame medical image data one by one in a sequential manner over a digital imaging and communications in medicine (DICOM) communications session with the image acquisition device.

19. The system of claim 18, wherein:
the plurality of images of the subject comprise an imaged procedure;
the agent module continues to receive from the image acquisition device the frames of the multi-frame medical image data one by one in the sequential manner until all the frames of the plurality of images have been received; and
the agent module compresses the multi-frame medical data to form the compressed data including a single data package with all the plurality of images of the subject for the imaged procedure.

20. The system of claim 18, wherein the agent module:
caches the frames of the multi-frame medical image data at the agent module; and
after receiving a DICOM storage commitment request from the image acquisition device indicating that all of the plurality of images have been sent:
compresses, at the agent module, the multi-frame medical image data to form the compressed data; and
sends a return confirmation message to the image acquisition device informing to delete local copies of the multi-frame medical image data.
